(19) United States
(12) Patent Application Publication
(10) Pub. No.: US 2005/0004079 A1
(43) Pub. Date: Jan. 6, 2005

(54) Pharmaceutical compositions for intranasal administration of [2-(8,9-dioxo-2,6-diazabicyclo[5.2.0]non-1(7)-en-2-yl)alkyl] phosphonic acid and derivatives and methods of use thereof

(75) Inventors: Eric J. Benjamin, Jamestown, NC (US); Reinhardt B. Baudy, Buckingham, PA (US); Michael R. Brandt, Flemington, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/820,215

(22) Filed: Apr. 7, 2004

Related U.S. Application Data
(60) Provisional application No. 60/461,571, filed on Apr. 9, 2003.

(51) Int. Cl. A61K 31/675

(52) U.S. Cl. 514/80

(57) ABSTRACT

Pharmaceutical compositions for intranasal administration are provided that contain at least one compound of formula (I) or a pharmaceutically acceptable salt thereof: 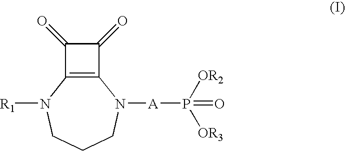 and one or more pharmaceutically acceptable additives for forming a composition for intranasal administration. Also provided are methods of treating one or more conditions in a mammal associated with a glutamate abnormality that includes administering intranasally to a mammal a therapeutically effective amount of a compound of formula (I) or a pharmaceutically acceptable salt thereof.

### CROSS-REFERENCE TO RELATED APPLICATIONS

This application claims the benefit under 35 U.S.C. §119(e) to U.S. provisional application 60/461,571, filed Apr. 9, 2003, which is hereby incorporated by reference in its entirety.

### BACKGROUND OF THE INVENTION

The present invention relates to intranasal compositions for administering [2-(8,9-dioxo-2,6-diazabicyclo[5.2.0]non-1 (7)-en-2-yl)alkyl]phosphonic acid and derivatives thereof, and methods of use thereof.

Glutamate and aspartate play dual roles in the central nervous system as essential amino acids and as the principal excitatory neurotransmitters. There are at least four classes of excitatory amino acid receptors: NMDA, AMPA (2-amino-3-(methyl-3-hydroxyisoxazol-4-yl)propanoic acid), kainate and metabotropic receptors. These excitatory amino acid receptors regulate a wide range of signaling events that impact physiological brain functions. For example, activation of the NMDA receptor has been shown to be the central event which leads to excitotoxicity and neuronal death in many disease states, as well as a result of hypoxia and ischaemia following head trauma, stroke and following cardiac arrest. It is also known that the NMDA receptor plays a major role in the synaptic plasticity that underlies many higher cognitive functions, such as memory and learning, certain nociceptive pathways, and in the perception of pain. In addition, certain properties of NMDA receptors suggest that they may be involved in the information-processing in the brain which underlies consciousness itself.

NMDA receptors are localized throughout the central nervous system. NMDA receptors are ligand-gated cation channels that modulate sodium, potassium and calcium ions flux when they are activated by glutamate in combination with glycine. Structurally, the NMDA receptor is thought to be comprised of heteromultimeric channels containing two major subunits designated as NR1 and NR2. These subunits contain a glycine binding site, a glutamate binding site and polyamine binding site. For the NR1 subunit, multiple splice variants have been identified, whereas for the NR2 subunit, four individual subunit types (NR2A, NR2B, NR2C, and NR2D) have been identified. The NMDA receptor also contains an Mg⁺⁺ binding site located inside the pore of the ionophore of the NMDA receptor/channel complex, which blocks the flow of ions.

Substantial preclinical and clinical evidence indicates that inhibitors of the N-methyl-D-aspartate (NMDA) receptor have therapeutic potential for treating numerous disorders. Disorders believed to be responsive to inhibition of NMDA receptors include cerebral vascular disorders such as cerebral ischemia (e.g., stroke) or cerebral infarction resulting in a range of conditions such as thromboembolic or hemorrhagic stroke, or cerebral vasospasm; cerebral trauma; muscular spasm; and convulsive disorders such as epilepsy or status epilepticus. NMDA receptor antagonists may also be used to prevent tolerance to opiate analgesia or to help control symptoms of withdrawal from addictive drugs.

Screening of compounds in recent years have identified a number of NMDA receptor antagonists that have been used in animal and clinical human studies to demonstrate proof of concept for the treatment of a variety of disorders. The difficulty with demonstrating clinical utility of NMDA receptor antagonists has generally been the antagonists' lack of NMDA receptor subtype selectivity and/or biological activity when dosed orally. The present invention provides intranasal compositions containing [2-(8,9-dioxo-2,6-diazabicyclo[5.2.0]non-1 (7)-en-2-yl)alkyl]phosphonic acid or derivatives thereof and methods of use thereof. The compounds useful in the present invention are NMDA antagonists, and as described in further detail herein have improved bioavailability when administered intranasally in comparison to oral administration.

### SUMMARY OF INVENTION

In one embodiment, the present invention provides a pharmaceutical composition for intranasal administration containing:
a) a therapeutically effective amount of at least one compound of formula (I) or a pharmaceutically acceptable salt thereof: 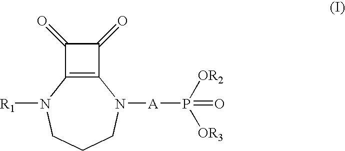
where:

R₁ is hydrogen, a C₁ to C₆ alkyl group, a C₂ to C₇ acyl group, a C₁ to C₆ alkanesulfonyl group, or a C₆ to C₁₄ aroyl group;
A is alkylene of 1 to 4 carbon atoms or alkenylene of 2 to 4 carbon atoms;
R₂ and R₃ are independently selected from hydrogen, or 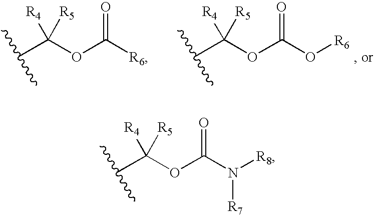
R₄ and R₅ are independently selected from hydrogen, a C₁ to C₄ alkyl group, a C₅ to C₇ aryl group, a C₆ to C₁₅ alkylaryl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety; a 5 to 13 membered heteroaryl group, a 6 to 21 membered alkylheteroaryl group having 5 to 13 members in the heteroaryl moiety, a C₄ to C₈ cycloalkyl group, a C₅ to C₁₆ alkylcycloalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety, a 5 to 13 membered heteroaryl group, a 6 to 21 membered alkylheteroaryl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted with 1 to about 5 substituents independently selected from a halogen atom, a cyano, nitro or hydroxyl group, a C₁-C₆ alkyl group, or a C₁-C₆ alkoxy group; and
b) one or more pharmaceutically acceptable additives for forming a composition for intranasal administration.

In another embodiment of the present invention, a pharmaceutical composition for intranasal administration, in unit dosage or multiple dose form, is provided that includes a therapeutically effective unit dosage or multiple dose for intranasal administration of at least one compound of formula (I), and one or more pharmaceutically acceptable additives for forming a composition for intranasal administration.

In yet another embodiment, the present invention provides a method for treating one or more conditions in a mammal that includes administering (preferably intranasally) to a mammal in need thereof a therapeutically effective amount of a compound of formula (I) or a pharmaceutically acceptable salt thereof. Examples of conditions that may be treated in accordance with the methods of the present invention include cerebral vascular disorders such as cerebral ischemia or cerebral infarction; cerebral trauma; muscular spasm; convulsive disorders such as epilepsy or status epilepticus; glaucoma; pain; anxiety disorders; mood disorders; schizophrenia; schizophreniform disorder; schizoaffective disorder; cognitive impairment; chronic neurodegenerative disorders such as Parkinson's disease, Huntingdon's disease, Alzheimer's disease, amyotrophic lateral sclerosis, or chronic dementia; inflammatory diseases; hypoglycemia; diabetic end organ complications; cardiac arrest; asphyxia anoxia; spinal chord injury; fibromyalgia, complications from herpes zoster (shingles) such as prevention of post-herpetic neuralgia; prevention of tolerance to opiate analgesia; or withdrawal symptoms from addictive drugs or combinations thereof.

### BRIEF DESCRIPTION OF THE DRAWINGS

FIG. 1 shows mean concentration levels of [2-(8,9-dioxo-2,6-diazabicyclo[5.2.0]non-1 (7)-en-2-yl)ethyl]phosphonic acid (Compound A) in monkey blood (sample size=4) versus time after compositions of the present invention were administered intranasally.

### DETAILED DESCRIPTION OF INVENTION

In one embodiment, the present invention provides pharmaceutical compositions for intranasal administration. The pharmaceutical composition of the present invention may be in any form suitable for intranasal administration. Examples of suitable forms include liquid forms such as solutions, gels, suspensions, dispersions, or emulsions and solid forms such as powders. The pharmaceutical compositions of the present invention have a pH ranging from 3 to 9, more preferably from about 4 to 8, and most preferably from about 6.5 to 7.5.

The pharmaceutical compositions of the present invention contain a therapeutically effective amount of at least one compound of formula I or a pharmaceutically acceptable salt thereof: 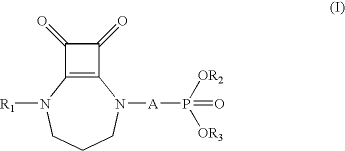
and one or more pharmaceutically acceptable additives for forming a composition for intranasal administration.

In formula (I) above:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylene of 1 to 4 carbon atoms or alkenylene of 2 to 4 carbon atoms;
R₂ and R₃ are independently selected from hydrogen, or 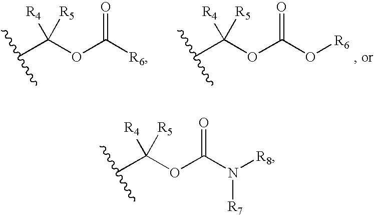
R₄ and R₅ are independently selected from hydrogen, a C₁ to C₄ alkyl group, a C₅ to C₇ aryl group, a C₆ to C₁₅ alkylaryl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety; a 5 to 13 membered heteroaryl group, a 6 to 21 membered alkylheteroaryl group having 5 to 13 members in the heteroaryl moiety, a C₄ to C₈ cycloalkyl group, a C₅ to C₁₆ alkylcycloalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety, a 5 to 13 membered heteroaryl group, a 6 to 21 membered alkylheteroaryl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur.

Unless otherwise indicated:

Alkyl or alkylene as used herein, refers to an aliphatic hydrocarbon chain having 1 to 12 carbon atoms and includes, but is not limited to, straight or branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 3 carbon atoms. In some embodiments of the invention, alkyl is preferably C₁ to C₈ and more preferably C₁ to C₆.

Alkenyl or alkenylene refers to an aliphatic straight or branched hydrocarbon chain having 2 to 7 carbon atoms that may contain 1 to 3 double bonds. Examples of alkenylene for A are straight or branched mono-, di-, or polyunsaturated groups such as vinyl, prop-1-enyl, allyl, methallyl, but-1-enyl, but-2-enyl or but-3-enyl.

Alkynyl refers to an aliphatic, straight or branched, hydrocarbon chain having 2 to 7 carbon atoms that may contain 1 to 3 triple bonds.

Acyl, as used herein, refers to the group R—C(═O)— where R is an alkyl group of 1 to 6 carbon atoms. For example, a C₂ to C₇ acyl group refers to the group R—C(═O)— where R is an alkyl group of 1 to 6 carbon atoms.

Alkanesulfonyl, as used herein, refers to the group R—S(O)₂— where R is an alkyl group of 1 to 6 carbon atoms.

Aryl, as used herein, refers to an aromatic 5- to 13-membered mono- or bi-carbocyclic ring such as phenyl or napthyl. Preferably, groups containing aryl moieties are monocyclic having 5 to 7 carbon atoms in the ring. Heteroaryl means an aromatic 5- to 13-membered carbon containing mono- or bi- cyclic ring having one to five heteroatoms which independently may be nitrogen, oxygen or sulfur. Preferably, groups containing heteroaryl moieties are monocyclic having 5 to 7 members in the ring where one to two of the ring members are selected independently from nitrogen, oxygen or sulfur. Groups containing aryl or heteroaryl moieties may optionally be substituted as defined below or unsubstituted.

Aroyl, as used herein, refers to the group Ar—C(═O)— where Ar is aryl as defined above. For example, a C₆ to C₁₄ aroyl moiety refers to the group Ar—C(═O)—where Ar is an aromatic 5 to 13 membered carbocylic ring.

Alkylaryl, as used herein refers to the group —R—Ar where Ar is aryl as defined above and R is an alkyl moiety having 1 to 8, preferably 1 to 6, and more preferably 1 to 4 carbon atoms. Examples of alkylaryl groups include benzyl, phenethyl, 3-phenylpropyl, and 4-phenyl butyl. Alkylheteroaryl, as used herein refers to the group —R-hetAr where hetAr is heteroaryl as defined above and R is an alkyl moiety having 1 to 8, preferably 1 to 6, and more preferably 1 to 4 carbon atoms.

Cycloalkyl, as used herein refers to a monocarbocyclic ring having 3 to 8 carbon atoms, e.g., cyclopropyl, cyclobutyl, cyclopentyl or cyclohexyl. Heterocycloalkyl refers to a carbon containing monocyclic ring having 3 to 8 ring members where one to two ring atoms are independently selected from nitrogen, oxygen or sulfur. Groups containing cycloalkyl or heterocycloalkyl moieties may optionally be substituted as defined below or unsubstituted.

Alkylcycloalkyl, as used herein, refers to the group —R-cycloalk where cycloalk is a cycloalkyl group as defined above and R is an alkyl moiety having 1 to 8, preferably 1 to 6, and more preferably 1 to 4 carbon atoms.

Halogen means fluorine, chlorine, bromine or iodine.

Pharmaceutically acceptable, as used herein, means a substance that is acceptable for use in pharmaceutical applications from a toxicological perspective and does not adversely interact with the active ingredient.

Substituted, as used herein, refers to a moiety, such as an aryl, heteroaryl, cycloalkyl or heterocycloalkyl moiety having from 1 to about 5 substituents, and more preferably from 1 to about 3 substituents independently selected from a halogen atom, a cyano, nitro or hydroxyl group, a C₁-C₆ alkyl group, or a C₁-C₆ alkoxy group. Preferred substituents are a halogen atom, a hydroxyl group, or a C₁-C₆ alkyl group.

In one embodiment of the present invention R₁ of formula I is preferably H or a C₁ to C₄ alkyl group and more preferably H.

In another embodiment of the present invention A of formula I is preferably an alkylene group, —(CH₂)ₙ—, where n is 1 to 3, more preferably 1 to 2 and most preferably 2.

In another embodiment, when it is desired to form a derivative of [2-(8,9-dioxo-2,6-diazabicyclo[5.2.0]non-1 (7)-en-2-yl)alkyl]phosphonic acid, preferably at least one of R₂ and R₃ is not H.

In other embodiments, R₂ and R₃ are preferably independently selected from H or:

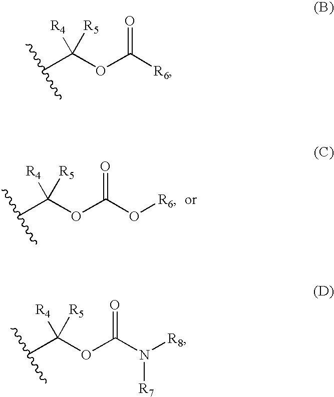

In another preferred embodiment of the present invention, R₂ and R₃ of formula (I) are H or the moiety (B) or (D), 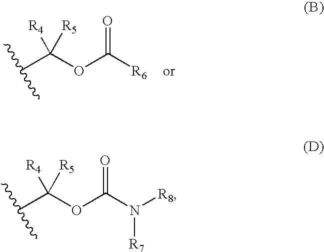
more preferably H or the moiety (B), and most preferably both are the moiety (B), where R₄, R₅ and R₆ are defined as above. When both R₂ and R₃ are not hydrogen, it is preferred that they be the same.

In another preferred embodiment of the present invention, both R₂ and R₃ are preferably hydrogen. When both R₂ and R₃ are hydrogen, it is most preferred that R₁ is hydrogen and A is ethylene (i.e., —(CH₂)₂—) to form the compound [2-(8,9-dioxo-2,6-diazabicyclo[5.2.0]non-1 (7)-en-2-yl)ethyl]phosphonic acid.

With respect to the moieties (B), (C), and (D), R₄ and R₅ are preferably selected from H or a C₁ to C₄ alkyl group, and more preferably H or methyl. R₆ is preferably selected from a C₃ to C₁₀ linear or branched alkyl group, a C₅ to C₇ aryl group, a 5- to 7-membered heteroaryl group, or a cycloalkyl group having in the ring 5 to 7 carbon atoms. In a preferred embodiment R₆, is a C₅ to C₇ aryl group.

In yet another preferred embodiment of the present invention R₁ is H or a C₁ to C₄ alkyl group; A is an alkylene group having the formula —(CH₂)ₙ—, where n is 1 to 3; R₂ and R₃ are independently selected from H or: 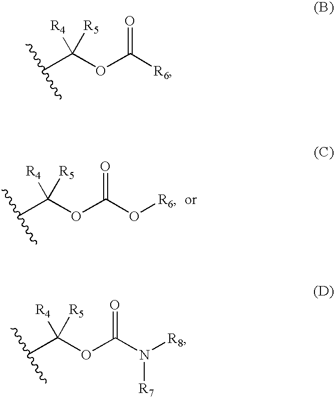
R₄ and R₅ are independently selected from H or a C₁ to C₄ alkyl group; and R₆ is selected from a C₃ to C₁₀ linear or branched alkyl group, a C₅ to C₇ aryl group, a 5- to 7-membered heteroaryl group, or a cycloalkyl group having in the ring 5 to 7 carbon atoms. In further embodiments, R₆ is selected from isopropyl, t-butyl, n-hept-4-yl, cyclohexyl and phenyl. In still further embodiments, R₇ and R₈ are both methyl.

Specific examples of compounds useful in the present invention include the following compounds:
[2-(8,9-dioxo-2,6-diazabicyclo[5.2.0]non-1 (7)-en-2-yl)ethyl]phosphonic acid;
3-{2-[8,9-dioxo-2,6-diazabicyclo[5.2.0]non-1 (7)-en-2-yl]ethyl}-3-oxido-7-oxo-7-phenyl-2,4,6-trioxa-3-phosphahept-1-yl benzoate;
3-{2-[8,9-dioxo-2,6-diazabicyclo[5.2.0]non-1 (7)-en-2-yl]ethyl}-3-oxido-7-oxo-8-propyl-2,4,6-trioxa-3-phosphaundec-1-yl 2-propylpentanoate;
2,2-dimethyl-propionic acid (2,2-dimethyl-propionyloxymethoxy)-[2-(8,9-dioxo-2,6-diaza-bicyclo[5.2.0]non-1 (7)-en-2-yl)-ethyl]-phosphinoyloxymethyl ester;
7-cyclohexyl-3-{2-[8,9-dioxo-2,6-diazabicyclo[5.2.0]non-1 (7)-en-2-yl]ethyl}-1,5-dimethyl-3-oxido-7-oxo-2,4,6-trioxa-3-phosphahept-1-yl cyclohexanecarboxylate;
7-cyclohexyl-3-{2-[8,9-dioxo-2,6-diazabicyclo[5.2.0]non-1 (7)-en-2-yl]ethyl}-3-oxido-7-oxo-2,4,6-trioxa-3-phosphahept-1-yl cyclohexanecarboxylate;
[2-(8,9-Dioxo-2,6-diaza-bicyclo[5.2.0]non-1-(7)-en-2-yl)-ethyl]-phosphonic acid diisopropoxycarbonyl oxymethyl ester;
[2-[8,9-dioxo-2,6-diazabicyclo[5.2.0]non-1 (7)-en-2-yl]ethyl]-phosphonic acid bis[1-(benzoyloxy)ethyl] ester;
benzoic acid [2-(8,9-dioxo-2,6-diaza-bicyclo[5.2.0]non-1 (7)-en-2-yl)-ethyl]-hydroxy-phosphinoyloxymethyl ester; and pharmaceutically acceptable salts thereof; and
[2-(8,9-Dioxo-2,6-diaza-bicyclo[5.2.0]non-1 (7)-en-2-yl)-ethyl]-phosphonic acid di-dimethylcarbamoyloxymethyl ester; and
pharmaceutically acceptable salts thereof.

The compounds useful in this invention may contain asymmetric carbon atoms and/or phosphorus atoms, and thus can give rise to optical isomers and diastereoisomers. While shown without respect to stereochemistry in formula (I), the present invention includes such optical isomers and diastereoisomers; as well as the racemic and resolved, enantiomerically pure R and S stereoisomers; as well as other mixtures of the R and S stereoisomers and pharmaceutically acceptable salts thereof.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In preferred embodiments, the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

One skilled in the art will also recognize that it is possible for tautomers to exist of formula (I). The present invention includes the use of all such tautomers even though not shown in formula (I).

The compounds useful in the present invention also include pharmaceutically acceptable salts of the compounds of formula (I). By “pharmaceutically acceptable salt”, it is meant any compound formed by the addition of a pharmaceutically acceptable base and a compound of formula (I) to form the corresponding salt. By the term “pharmaceutically acceptable” it is meant a substance that is acceptable for use in pharmaceutical applications from a toxicological perspective and does not adversely interact with the active ingredient. Preferably, the pharmaceutically acceptable salts are alkali metal (sodium, potassium, lithium) or alkaline earth metal (calcium, magnesium) salts of the compounds of formula (I), or salts of the compounds of formula (I) with pharmaceutically acceptable cations derived from ammonia or a basic amine. Examples of the later include, but are not limited to, ammonium, mono-, di-, or trimethylammonium, mono-, di-, or triethylammonium, mono-, di-, or tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 1-isopropylpyrrolidinium, 1,4-dimethylpiperazinium, 1-n-butylpiperidinium, 2-methylpiperidinium, 1-ethyl-2-methylpiperidinium, mono-, di-, or triethanolammonium, tris-(hydroxymethyl)methylammonium, or phenylmonoethanolammonium. Preferably, salts may be formed when at least one of R₂ or R₃ is hydrogen.

The compounds useful in the present invention can be prepared by synthesizing the compound of the formula (II), where A and R₁ are defined as for formula (I) 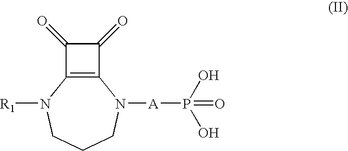
according to methods described in U.S. Pat. Nos. 5,168,103, 5,240,946, 5,990,307 and 6,011,168, the contents of which are entirely incorporated herein by reference. A preferred synthesis route is described in Example 5 of U.S. Pat. Nos. 5,990,307 and 6,011,168.

To form compounds where at least one of R₂ or R₃ is not hydrogen in formula (I), the compound of formula (II) obtained is dissolved in a suitable solvent such as dimethylformamide. By “suitable solvent” it is meant a solvent that the compound of formula (II) is soluble in and nonreactive with. Preferably an acid scavenger (to react with the acid halide reaction by-product) such as an amine, is added to the reaction mixture at preferably ambient temperature. The amine is preferably a sterically hindered secondary or tertiary amine and more preferably a tertiary amine such as diisopropylethylamine. An appropriately substituted ester of the formula: 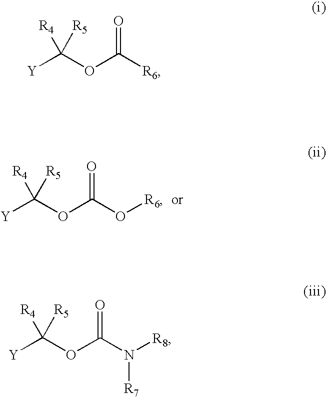
where R₄, R₅, and R₆ are defined as in formula (I), and Y is a leaving group, is added to the reaction mixture. As used herein, the term “leaving group” refers to a moiety that can be selectively displaced by another moiety, such as by nucleophilic substitution or elimination, during a chemical reaction. Typically, leaving groups include moieties that when removed by nucleophilic substitution or elimination are relatively stable in anionic form. Leaving groups are well known in the art and include, for example, halides (e.g., chloride, bromide, and iodide) and alkyl- and arylsulfonates such as mesylate, tosylate, brosylate, nosylate, triflate, and the like. In a preferred embodiment, Y is a halogen atom.

The reaction mixture is heated from about 50° C. to about 80° C., and more preferably from about 65° C. to about 75° C. for a sufficient reaction time so that the halo ester reacts with the compound of formula (II) to form a compound of formula (I). Typically, for preferable yields, the reaction time is from about 20 hours to about 40 hours, and more preferably from about 25 hours to about 35 hours. After the reaction is complete, the reaction mixture is preferably cooled to ambient temperature, and the compound of formula (I) is isolated using standard techniques known to those skilled in the art. A preferred isolation method is to partition the reaction mixture between a mild base, such as aqueous sodium bicarbonate, and an organic solvent such as ethyl acetate. The aqueous phase is preferably several times re-extracted with the organic solvent, and the combined organic layers are washed again with a mild base. The organic layers are then dried, for example with brine and over magnesium sulfate, filtered and evaporated. The residue is then preferably flash chromatographed on silica gel using standard techniques to isolate the compound. Further details concerning the compounds and their synthesis, where at least one of R₂ or R₃ is not hydrogen in formula (I), can be found in U.S. provisional application Ser. No. 60/461,490, filed on Apr. 9, 2003, and U.S. application Ser. No., not yet assigned, filed concurrently with this application, entitled “Derivatives Of [2-(8,9 -Dioxo-2,6-Diazabicyclo[5.2.0]Non-1(7)-en-2-yl)Alkyl] Phosphonic Acid And Methods Of Use Thereof”, the disclosures of which are incorporated herein by reference in their entireties.

The compound of formula (I) is present in the intranasal composition in a therapeutically effective amount for intranasal administration. As used herein “a therapeutically effective amount” is at least the minimal amount of the compound of formula (I) or a pharmaceutically acceptable salt form thereof, which treats the condition in question in a mammal. The therapeutically effective amount will depend on such variables as the particular composition used, the severity of the symptoms, and the particular patient being treated. To determine the therapeutically effective amount of the compound to be administered, the physician may, for example, evaluate the effects of a given compound of formula (I) in the patient by incrementally increasing the dosage until the desired symptomatic relief level is achieved. The continuing dose regimen may then be modified to achieve the desired result. For intranasal administration, preferably the compounds of the present invention are incrementally increased in a patient in an amount of from 1 mg/kg to 10 mg/kg until the desired symptomatic relief level is achieved. The continuing dose regimen may then be modified to achieve the desired result, with the range for intranasal dosage being preferably from about 200 mg/day to about 600 mg/day.

The intranasal pharmaceutical composition of the present invention, in addition to containing a therapeutically effective amount of at least one compound of formula (I), contains one or more pharmaceutically acceptable additives for forming a composition for intranasal administration. By “one or more pharmaceutically acceptable additives for forming a composition for intranasal administration” it is meant one or more substances that facilitate delivery of the compound of formula (I) by intranasal administration. Examples of pharmaceutically acceptable additives for forming a composition for intranasal administration include liquid or solid carriers; absorbance enhancers; pH adjusting agents; buffers; metal chelating agents; thickening agents; humectants; or bioadhesives or combinations thereof. Preferably, these additives in total will constitute at least about 0.25 weight percent, more preferably from about 0.25 weight percent to about 95 weight of the composition, based on the total weight of the composition.

If the composition is a liquid, the composition will contain preferably from about 50 to about 95 and more preferably from about 70 to about 95 weight percent of one or more liquid carriers, based on the total weight of the composition. Examples of liquid carriers include water, or a mixture of water and one or more other pharmaceutically acceptable solvents, such as, alcohol, propylene glycol, glycerin or combinations thereof. In a preferred embodiment, the liquid carrier is aqueous based (preferably at least about 70 weight percent water and more preferably at least about 85 weight percent water, based on the total weight of the liquid carrier) and most preferably water.

If the composition is a powder, the composition may optionally contain from 0 to about 50 weight percent, and more preferably from about 0.10 weight percent to about 20 weight percent of one or more solid carriers, based on the total weight of the composition. Examples of solid carriers include water soluble polymers such as povidones, polyvinyl alcohol or hydroxypropyl methylcellulose, or water insoluble polymers, such as, microcrystalline cellulose or sugars such as sucrose, mannitol, dextrose, or lactose.

Absorbance enhancers are additives that enhance the absorbance of compounds of formula (I). Preferably, one or more absorbance enhancers may optionally be present in the composition in an amount of from about 0.2 weight percent to about 2 weight percent and more preferably from about 0.5 weight percent to about 1 weight percent, based on the total weight of the composition. Examples of absorbance enhancers include surfactants such as sodium lauryl sulfate or polysorbates; bile salts such as cholates or glycocholates; fusidic acid derivatives; fatty acids and salts such as oleic acid or sodium caprate; chelating agents such as ethylenediamine tetraacetic acid (EDTA) or combinations of these ingredients.

One or more agents for adjusting the pH such as inorganic or organic bases may optionally be present in the composition to bring the pH of the composition within the range of 3 to 9, more preferably from about 4 to about 8 and most preferably from about 6.5 to about 7.5. Examples of suitable inorganic bases include ammonium hydroxide, or alkali or alkaline earth metal hydroxides such as sodium hydroxide or potassium hydroxide. Examples of suitable organic bases that may be used include ethanolamine or triethanolamine.

In addition to pH adjusting agents, the composition of the present invention may optionally contain one or more pharmaceutically acceptable buffers such as acetates, citrates, phosphates, or trolamine or combinations thereof.

The pharmaceutical composition may also optionally contain metal chelating agents such as ethylene diamine tetraacetic acid (EDTA). Preferably, the metal chelating agents, if desired, are present in an amount of from about 0.005 weight percent to about 0.5 weight percent and more preferably from about 0.05 weight percent to about 0.2 weight percent, based on the total weight of the composition.

To increase residence time in the nasal cavity, the viscosity of the pharmaceutical composition may be increased by incorporation of one or more thickening agents. Examples of suitable thickening agents include cellulose based polymers such as methyl cellulose, hydroxypropylmethylcellulose, hydroxypropylethylcellulose, or hydroxypropylcellulose; chitosan; xanthan gums; or povidone or combinations thereof. Although the concentration of the thickening agent will depend upon the thickening agent used and the desired viscosity, preferably, the amount of the one or more thickening agents in the composition will range from 0 to about 5 weight percent and more preferably from about 0.1 to about 2 weight percent, based on the total weight of the composition.

The composition may also optionally contain one or more humectants to keep the mucous membrane moist and to reduce irritation. Examples of suitable humectants useful in the present invention include sorbitol, propylene glycol, or glycerol, or combinations thereof. Although the concentration of the humectant in the composition will depend upon the agent used, preferably the total amount of humectant, if present in the composition, will range from about 0.1 weight percent to about 20 weight percent and more preferably from about 1 weight percent to about 5 weight percent, based on the total weight of the composition.

The composition may also contain one or more bioadhesives to increase residence time in the nasal cavity. Examples of bioadhesives useful in the present invention include methyl cellulose, carbomer, carboxymethyl cellulose, starches, hyaluronates and chitosans. Although the concentration of the bioadhesive in the composition will depend upon the agent used, preferably the total amount of bioadhesive, if present in the composition, will range from about 0.1 weight percent to about 5 weight percent, based on the total weight of the composition.

The intranasal pharmaceutical compositions of the present invention may also optionally contain one or more antimicrobial preservatives to prevent microbial growth during storage and multiple dose use. Examples of suitable preservatives are benzalkonium chloride, thimersal, chlorobutanol, or parabens, or combinations thereof. Although the concentration of the preservative in the composition will depend upon the preservative used, preferably the total amount of preservative present in the composition will range from about 0.1 weight percent to about 2.0 weight percent, based on the total weight of the composition.

Examples of other liquid or solid carriers; absorbance enhancers, pH adjusting agents, buffers, thickening agents, humectants, bioadhesives or antimicrobial preservatives, or combinations thereof may be found in for example those texts known to those skilled in the art such as Remington: The Science and Practice of Pharmacy, 18^{th} Edition, ed. Alfonoso R. Gennaro, Mack Publishing Company, Easton, Pa. (1995); and Kibbe, A. R. (Ed.), Hand Book of Pharmaceutical Excipients. American Pharmaceutical Association, 3rd Edition (2000). Additionally, other pharmaceutically acceptable additives and specific liquid or solid carriers; absorbance enhancers, pH adjusting agents, buffers, thickening agents, humectants, bioadhesives or antimicrobial preservatives for intranasal administration may be found in Behl, C. R., et. al., Optimization of Systemic Nasal Drug Delivery With Pharmaceutical Excipients, Advanced Drug Delivery Reviews, 29, 117-133, (1998); and Zia, H., et. al., Intranasal Drug Delivery. Clinical Research and Regulatory Affairs, 10(2), 99-135 (1993), the disclosures of which are hereby incorporated by reference in their entireties.

In one preferred embodiment of the present invention, the pharmaceutical composition is in the form of a liquid. The liquid composition is preferably in the form of a solution. For liquid compositions, the amount of compound of formula I is preferably present in an amount of about 10 mg/ml to about 500 mg/ml, and more preferably from about 50 mg/ml to about 300 mg/ml. The liquid composition is also preferably aqueous based. Preferably, the amount of water present in the liquid composition is preferably from about 50 weight percent to about 99 weight percent and more preferably from about 70 weight percent to about 90 weight percent, based on the total weight of the composition. The liquid composition will also preferably contain one or more pH adjusting agents to adjust the pH from about 3 to about 9 and more preferably from about 4 to about 8. The viscosity of the liquid formulation preferably ranges from about 2 cps to about 8 cps, and more preferably from about 4 to about 6 cps as measured by Oswald Viscometer.

In another preferred embodiment of the present invention, the pharmaceutical composition is in the form of a powder. Preferably, the powder will have a particle size of less than about 250 micron (e.g., all particles passing through a 250 micron screen) and more preferably less than about 180 micron as measured by sieve analysis. The amount of compound of formula I in the powder formulation will preferably be from about 50 weight percent to about 99.75 weight percent and more preferably from about 70 weight percent to about 90 weight percent, based on the total weight of the formulation. When the composition is a powder it can be formed into a solution having a pH from about 3 to about 9, more preferably from about 4 to about 8, most preferably from about 6.5 to about 7.5.

In another embodiment of the present invention, the pharmaceutical composition may contain one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

A more complete listing of pharmaceutical active agent can be found in the Physicians' Desk Reference, 55 Edition, 2001, published by Medical Economics Co., Inc., Montvale, N.J. Each of these agents may be administered according to the therapeutically effective dosages and regimens known in the art, such as those described for the products in the Physicians' Desk Reference, 55 Edition, 2001, published by Medical Economics Co., Inc., Montvale, N.J.

In another embodiment of the present invention, the pharmaceutical composition is in unit dosage or multiple dose form. In such form, the composition is sub-divided in unit or multiple doses containing appropriate quantities of the active ingredient. The dosage forms can be packaged compositions, for example packeted powders, vials, ampoules, or sachets containing liquids. Thus, the present invention also provides a pharmaceutical composition in unit dosage or multiple dose form containing a therapeutically effective unit or multiple dosage for intranasal administration of at least one compound of formula (I), and one or more pharmaceutically acceptable additives for forming a composition for intranasal administration.

As one skilled in the art will recognize, the preferred effective unit or multiple dosage will depend on for example, the condition being treated and the particular compound chosen for formula 1. Preferably, however, a dosage (whether in unit or multiple dosage form) for intranasal administration will range from about 100 mg to about 700 mg and more preferably from about 200 mg to about 600 mg of the compound of formula I useful in the present invention.

In another embodiment of the present invention, the present invention provides methods for treating conditions associated with glutamate abnormalities that includes administering intranasally to a mammal in need thereof a therapeutically effective amount of at least one compound of formula (I). As used herein, “associated with” refers to conditions directly or indirectly caused by glutamate abnormalities. “Glutamate abnormality” refers to any condition produced by a disease or a disorder in which glutamate, typically in increased amounts, is implicated as a contributing factor to the disease or disorder. Conditions believed to be associated with glutamate abnormality include, but are not limited to, cerebral vascular disorders such as cerebral ischemia (e.g., stroke) or cerebral infarction resulting in a range of conditions such as thromboembolic or hemorrhagic stroke, or cerebral vasospasm; cerebral trauma; muscular spasm; convulsive disorders such as epilepsy or status epilepticus; glaucoma; pain; anxiety disorders such as such as panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; mood disorders such as bipolar disorders (e.g., bipolar I disorder, bipolar II disorder, and cyclothymic disorder), depressive disorders (e.g., major depressive disorder, dysthymic disorder, or substance-induced mood disorder), mood episodes (e.g., major depressive episode, manic episode, mixed episode, and hypomanic episode); schizophrenia; schizophreniform disorder; schizoaffective disorder; cognitive impairment such as memory loss; and chronic neurodegenerative disorders such as Parkinson's disease, Huntingdon's disease, Alzheimer's disease, amyotrophic lateral sclerosis, or chronic dementia related to, for example, Lewy body disease, Alzheimer's disease, fronto temporal, or AIDS. With respect to the mental disorders listed above such as schizophrenia, mood disorders and anxiety disorders, reference is made to the Diagnostic and Statistical Manual of Mental Disorders, 4^{th} edition, Washington, D.C., American Psychiatric Association (1994) for a more complete description of each of the mental disorder.

Additional conditions believed to be related to glutamate abnormalities include inflammatory diseases; hypoglycemia; diabetic end organ complications; cardiac arrest; asphyxia anoxia, such as from near drowning, pulmonary surgery and cerebral trauma; and spinal chord injury. The compounds of the present invention may also be used to treat fibromyalgia, and complications from herpes zoster (shingles) such as prevention of post-herpetic neuralgia. The compounds useful in the present invention may also be used to prevent tolerance to opiate analgesia or to help control symptoms of withdrawal from addictive drugs. Thus, the present invention provides methods for treating each of the aforementioned conditions that includes administering intranasally to a mammal in need thereof a therapeutically effective amount of at least one compound of formula (I).

In one preferred embodiment, the compounds useful in the present invention are used to treat pain. The pain may be, for example, acute pain (short duration) or chronic pain (regularly reoccurring or persistent). The pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain or dental pain, or headaches such as migraines or tension headaches, or combinations of these pains. One skilled in the art will recognize that these pains may overlap one another. For example, a pain caused by inflammation may also be visceral or musculoskeletal in nature.

In a preferred embodiment of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In a more preferred embodiment, the compounds useful in this invention are used to treat chronic pain that is neuropathic pain, visceral pain, musculoskeletal pain, bony pain, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns or central pain conditions related to thalamic conditions.

As mentioned previously, the methods of the present invention may be used to treat pain that is somatic and/or visceral in nature. For example, somatic pain that can be treated in accordance with the methods of the present invention include pains associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, the chronic pain may be with or without peripheral or central sensitization.

The compounds useful in this invention may also be used to treat acute and/or chronic pains associated with female conditions, which may also be referred to as female-specific pain. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

The compounds of the present invention may be administered neat (i.e., as is) or in an intranasal pharmaceutical composition containing one or more pharmaceutically acceptable additives for forming a composition for intranasal administration as previously described herein. In a preferred embodiment, the compounds useful in the present invention are administered in the form of an intranasal pharmaceutical composition as previously described herein.

In another embodiment of the present invention, the compounds useful in the present invention are administered using a pre-measured unit dosage dispenser. One skilled in the art will recognize that there are a variety of unit or multiple dosage dispensers that may be used, and the selection will depend on for example the compound and pharmaceutical composition being dispensed. For example, in the case of liquid compositions, dropper or spray devices may be used; in the case of powder compositions, dry powder inhalers may be used.

In another embodiment of the present invention, the compounds useful in the present invention may be administered to a mammal with one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The method of administration of the other pharmaceutical active agent may be the same or different from the route of administration used for the compounds of the present invention. For example, the other pharmaceutical active agents may be administered by oral or parental administration, such as for example, by intramuscular, intraperitoneal, epidural, intrathecal, intravenous, intramucosal such as by intranasal or sublingual, subcutaneous or transdermal administration. The preferred administration route will depend upon the particular pharmaceutical active agent chosen and its recommended administration route(s) known to those skilled in the art.

A more complete listing of pharmaceutical active agent can be found in the Physicians' Desk Reference, 55 Edition, 2001, published by Medical Economics Co., Inc., Montvale, N.J. Each of these agents may be administered according to the therapeutically effective dosages and regimens known in the art, such as those described for the products in the Physicians' Desk Reference, 55 Edition, 2001, published by Medical Economics Co., Inc., Montvale, N.J.

In a preferred embodiment of the present invention, the compounds useful in the present invention may be administered to a mammal with one or more other pain relieving agents to treat pain in a mammal. By “pain relieving agents” it is meant any agent that directly or indirectly treats pain symptoms. Examples of indirect pain relieving agents include for example anti-inflammatory agents, such as anti-rheumatoid agents.

The one or more other pain relieving agents may be administered simultaneously (such as individually at the same time, or together in a pharmaceutical composition), and/or successively with the compounds of the present invention. Preferably, the compounds of the present invention and the one or more pain relieving agents are administered in a manner so that both are present in the mammal body for a certain period of time to treat pain.

The method of administration of the other pain relieving agent may be the same or different from the route of administration used for the compound of the present invention. For example, opioids are preferably administered by oral, intravenous, intranasal, or intramuscular administration routes.

One skilled in the art will recognize that the dosage of the other pain relieving agent administered to the mammal will depend on the particular pain relieving agent in question and the desired administration route. Accordingly, the other pain relieving agent may be dosed and administered according to those practices known to those skilled in the art such as those disclosed in references such as the Physicians' Desk Reference, 55 Edition, 2001, published by Medical Economics Co., Inc., Montvale, N.J.

Examples of pain relieving agents that may be administered with the compound of the present invention include analgesics such as non-narcotic analgesics or narcotic analgesics; anti-inflammatory agents such as non-steroidal anti-inflammatory agents (NSAID), steroids or anti-rheumatic agents; migraine preparations such as beta adrenergic blocking agents, ergot derivatives, or isometheptene; tricyclic antidepressants such as amitryptyline, desipramine, or imipramine; anti-epileptics such as gabapentin, carbamazepine, topiramate, sodium valproate or phenyloin; α₂ agonists; or selective serotonin reuptake inhibitors/selective norepinepherine uptake inhibitors, or combinations thereof. One skilled in the art will recognize that some agents described hereinafter act to relieve multiple conditions such as pain and inflammation, while other agents may just relieve one symptom such as pain. A specific example of an agent having multiple properties is aspirin, where aspirin is anti-inflammatory when given in high doses, but at lower doses is just an analgesic. The pain relieving agent may include any combination of the aforementioned agents, for example, the pain relieving agent may be a non-narcotic analgesic in combination with a narcotic analgesic.

In a preferred embodiment of the present invention, at least one compound of the present invention is administered with at least one opioid analgesic in accordance with the methods previously described herein to treat pain. It has been found that the compounds of the present invention, when administered with at least one opioid analgesic such as morphine, have such beneficial effects as synergistically decreasing pain perception, increasing the duration of pain relief, and/or decreasing adverse side effects.

### EXAMPLES

The compounds of formula (I) useful in the present invention were evaluated for their effectiveness when administered intranasally.

The in vivo test methods used herein for evaluating pain have been used by others skilled in the art to evaluate the effectiveness of compounds for relieving pain. See e.g., Bennett GJ and Xie TK, A peripheral mononeuropathy in rat produces disorders of pain sensation like those seen in man, Pain 33: 87-107 (1988); Chaplan SR, Bach RW, Pogrel JW, Chung JM and Yaksh TL, Quantitative assessment of tactile allodynia in the rat paw, J. Neurosci. Methods 53: 55-63 (1994); and Mosconi T and Kruger L, Fixed-diameter polyethylene cuffs applied to the rat sciatic nerve induce a painful neuropathy: ultrastructural morphometric analysis of axonal alterations Pain 64: 37-57 (1996).

Synthesis of Compounds Used in the Examples

Compound A—[2-(8,9-dioxo-2,6-diazabicyclo[5.2.0]non-1(7)-en-2-yl)ethyl]phosphonic acid

Compound A was prepared according to the procedure described in U.S. Pat. No. 5,990,307, Example No. 5.

Compound B—3{2-[8,9-Dioxo-2,6-diazabicyclo[5.2.0 ]non-1 (7)-en-2-yl]ethyl}-3-oxido-7-oxo-7-phenyl-2,4,6-trioxa-3-phosphahept-1-yl benzoate

A solution of [2-(8,9-dioxo-2,6-diazabicyclo[5.2.0]non-1 (7)-en-2-yl)ethyl]phosphonic acid prepared according to the procedure described above (20.16 mmol, 5.25 g) in dry DMF (120 mL) was treated with N,N-diisopropylethylamine (80.64 mmol, 14 ml) for Y2 hour at ambient temperature. Benzoic acid chloromethyl ester (60.49 mmol, 10.32 g, synthesis described below) was added at ambient temperature under exclusion of moisture. The reaction mixture was heated to 65° C. for 20 hours. The temperature was then raised to 72° C. and stirred at 72° C. for 16 hours after which the reaction was completed. The mixture was cooled to room temperature and partitioned between 10% sodium bicarbonate and ethyl acetate. After separation of the layers the aqueous phase was again extracted with ethyl acetate (6×) until there was no more product in the water phase (by silica gel TLC, 7% 2M ammonia in methanol and 93% chloroform). The combined organic layers were washed with brine, dried over magnesium sulfate, filtered and evaporated to dryness. The residue was flash chromatographed on 400 g silica gel using a solvent mixture of 1% 2M ammonia in methanol and 99% chloroform. Gradually the percentage of ammonia in methanol was increased to 7% and 93% chloroform. The solvent was evaporated in vacuo to yield the desired product (10.5 g, 99%; glass like material). MS (ES−): m/e 527 (M−H).

Preparation of reactant benzoic acid chloromethyl ester:

Para-formaldehyde (4.5 g) and zinc chloride (catalytic amount) were mixed together at 0° C. Benzoyl chloride (0.142 mole, 20 g) was added dropwise over 1 hour. The reaction was warmed to ambient temperature, then was heated to 55° C. for 10 hours. The progress of the reaction was followed by TLC (silica gel, 5/95, ethyl acetate/hexane). Since the starting material was still seen, an additional 1 g para-formaldehyde was added. The reaction was continued stirring at 55° C. for an additional 10 hours, cooled and flash chromatographed on 500 g silica gel, eluting with a solvent mixture of 2% ethyl acetate and 98% hexane. The solvent was evaporated in vacuo. Since the product had a low boiling point, the rotovapor bath temperature was not above 35° C. The desired product, 11.82 g (49%) was obtained as clear oil. MS (ES+): m/e 171 (M+H).

Preparation of Pharmaceutical Compositions of the Present Invention

### Example 1

### Compound A Nasal Solution 300 mg/ml

The following composition was prepared as described below:

| | Amount | |
|---|---|---|
| Ingredients | (gm) | |
| Compound A | 30.00 | |
| EDTA | 0.10 | |
| NaOH solution (5N) | 37 | mL |
| Deionized Water | 50 | mL |
| Total | 100 | mL |

Ethylenediaminetetraacetic acid (EDTA) was dissolved in 50 ml deionized water with stirring. Compound A was added and dissolved with stirring and by addition of 5N sodium hydroxide solution. After compound A was completely dissolved and a pH of 7 was reached, the volume was made up to 100 ml with additional deionized water and the pH was adjusted to 7.01 with sodium hydroxide solution. 10 ml of the resulting solution was filled in a high density polyethylene (HDPE) bottle fitted with a metered dose nasal spray pump designed to administer 100 μl of nasal spray upon each actuation.

### Example 2

### Compound A Nasal Solution 50 mg/ml

The following composition was prepared as described below:

| | Amount | |
|---|---|---|
| Ingredients | (gm) | |
| Example 1 - 300 mg/ml | 10.0 | ml |
| solution | | |
| HPMC C15 LV | 0.45 | gm |
| DI Water QS | 60 | mL |
| Total | 60 | mL |

10 ml of the 300 mg/ml solution of Example 1 was diluted with 45 ml of deionized water. Hydroxypropylmethyl cellulose (HPMC C15 LV, supplied by Dow Chemicals) was added to this solution slowly and with stirring. The volume was made up to 60 ml with additional water. The pH of the solution was 7.00. 10 ml of the resulting solution was filled in a HDPE bottle fitted with a metered dose nasal spray pump designed to administer 100% of nasal spray upon each actuation.

### Example 3

### Compound A Sodium Powder 730 mg/gm

The following composition was prepared as described below:

| | Amount |
|---|---|
| Ingredients | (gm) |
| Compound A-Sodium salt | 3.504 |
| EDTA | 0.01 |
| Total | 3.514 |

The Compound A Nasal Solution 300 mg/ml was prepared as described in Example 1. 10 ml of this solution was transferred to a 50 ml round bottom flask and the water was evaporated under vacuum using a rotary evaporator (bath temperature 30° C.). The bath temperature was raised to 50° C. for additional drying. 15 ml of cold absolute alcohol was added to the powder in the flask and stirred for 15 minutes. The powder was separated by filtration, air dried to remove alcohol and then dried in an oven under vacuum for 2 hours. The final loss on drying was 3.52%. The pH of the powder when dissolved in deionized water (100 mg/5 ml) was 7.4, and the compound A content of the powder was 73.17%. 41 mg (equivalent to 60 mg of compound A in free acid form) of the powder was filled in a device for the intranasal administration of powder.

### Examples 4-8

### Evaluation of Intranasal Pharmaceutical Compositions

### Examples 4 to 7

### Intranasal Absorption Studies in Monkeys

Female Cynomolgus monkeys were fasted overnight. The compositions of Examples 1 to 3 were administered as shown in Table 1.

TABLE 1
Intranasal Administration of EAA-090 in Monkeys

| | | Dose of | Dose | |
|---|---|---|---|---|
| | | Composition | of Active | |
| Example | Composition | (μl) | (mg) | Delivery Method |
| 4 | Example 2 | 200 μl | 10 mg | 1 spray of 100 μl |
| | | | | in each nostril |
| 5 | Example 1 | 200 μl | 60 mg | 1 spray of 100 μl |
| | | | | in each nostril |
| 6 | Example 1 | 400 μl | 120 mg | 2 sprays of 100 μl |
| | | | | in each nostril |
| 7 | Example 3 | 82 mg | 60 mg* | 41 mg (30 mg*) |
| | | | | in each nostril |

*As Compound A free acid contents.

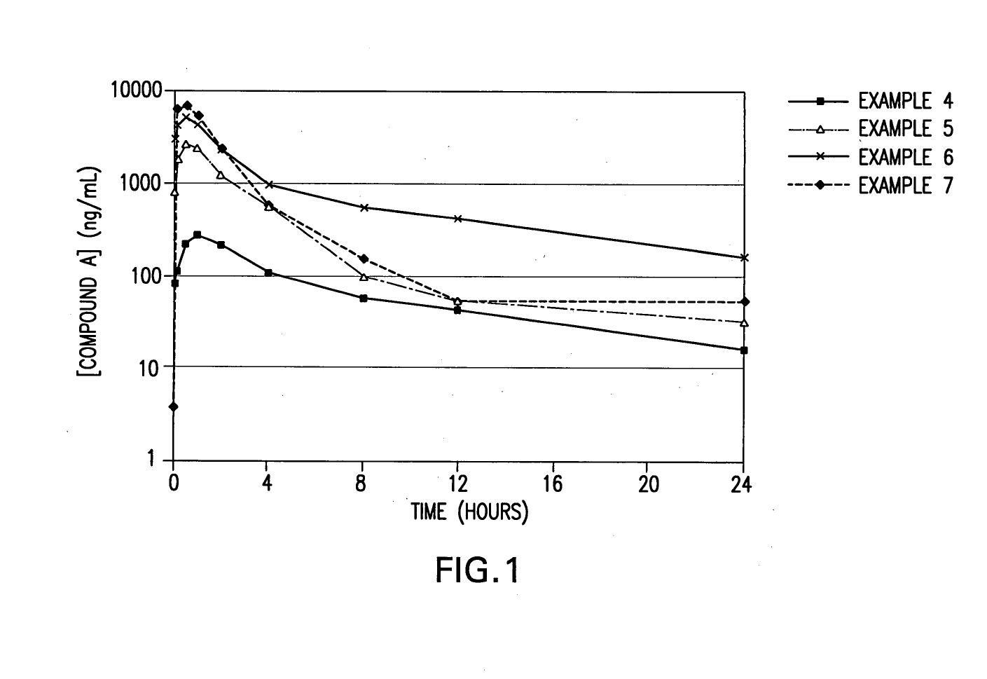

Blood samples were collected at various intervals and analyzed for active ingredient content (i.e., compound A). Compound A concentrations in blood versus time is shown in FIG. 1. The pharmacokinetics parameters are presented in Table 2, where AUC is the area under the EAA-090 blood concentration vs time (0-24 hours) curve, Cmax is the maximum concentration, and tmax is the time at which the maximum concentration occurred.

TABLE 2
Mean (SD)** Pharmacokinetic Parameters of Intranasal
Compositions in Monkeys (n = 4)

| | AUC₀₋₂₄ | C max | t max |
|---|---|---|---|
| Example | (μg · hr/mL) | (μg/mL) | (hr) |
| Example 4 | 1.66 (0.82)** | 0.28 (0.06) | 1.25 (0.50) |
| 10 mg Dose (˜3 mg/kg) | | | |
| Example 5 | 7.89 (5.59) | 2.96 (1.31) | 0.63 (0.25) |
| 60 mg Dose | | | |
| (˜18 mg/kg) | | | |
| Example 6 | 19.6 (5.62) | 5.07 (1.31) | 0.50 (0.00) |
| 120 mg Dose | | | |
| (˜36 mg/kg) | | | |
| Example 7 | 15.4 (1.96) | 7.50 (1.79) | 0.20 (0.20) |
| 60 mg Dose | | | |
| (˜18 mg/kg) | | | |

**Numbers in parenthesis are the standard deviation, sample size was 4.

From earlier studies the AUC in Monkeys after a 1.1 mg/kg IV dose was 1.67 ughr/ml. The AUC after a 20 mg/kg oral dose was 1.74 ug*hr/ml and the Cmax was 147 ng/ml. Compound A thus has an oral bioavailability of approximately 2.5% at a dose of 100 mg/kg in Monkeys. Bioavailabilities in this range have a potential of increasing the dose and the cost of the product.

Based on these IV and oral data, absolute bioavailabilities from intranasal administration of a solution and powder composition are approximately 14% and 22%, respectively. The total exposure from intranasal administration of the solution is 5-fold and the powder is 10-fold greater than from oral administration. The Cmax values from the intranasal solution and powder are about 20 and 50 fold higher than that from oral administration.

### Example 8

### In vivo Efficacy in Rats: Prostaglandin E₂-Induced Thermal Hypersensitivity.

Subjects: Individually housed Spraque-Dawley rats had free access to rat chow and water. A 12-h light/12-h dark cycle was in effect (lights on from 6:00 am to 6:00 pm). Animal maintenance and research were conducted in accordance with the guidelines provided by the National Institutes of Health Committee on Laboratory Animal Resources. These subjects were used in the tests below.

Procedure: The terminal 10 cm of the tail was placed into a thermos bottle containing water warmed to 38, 42, 46, 50 or 54° C. The latency in seconds for the animal to remove the tail from the water was used as a measure of nociception. If the animal did not remove the tail within 20 sec, the experimenter removed the tail and a maximum latency of 20 sec was recorded.

Following the assessment of baseline thermal sensitivity, thermal hypersensitivity was produced by a 50 μL injection of 0.1 mg prostaglandin E₂ (PGE₂) into the terminal 1 cm of the tail. Temperature-effect curves were generated before (baseline) and after (15, 30, 60, 90 and 120 min) the PGE₂ injection. Previous studies in other species (e.g., monkeys; Brandt et al., J. Pharmacol. Exper. Ther. 296:939, 2001) and results from the current study demonstrate that PGE₂ produces a dose- and time-dependent thermal hypersensitivity that peaks 15 min after injection and dissipates after 2 hr.

The ability of compounds to reverse PGE₂-induced thermal hypersensitivity was assessed using a single dose time-course procedure. Under this procedure, a single dose of the compound to be tested was administered intraperitoneally (IP), orally (PO) or intranasally (IN) 30 min before the injection of PGE₂. Tactile sensitivity was assessed 30 min after PGE₂ injection. For the IP and PO administration, compound was administered in a volume of 1 ml/kg with the dose administered calculated as mg/kg. For IN administration, rats were lightly anesthetized with 3.5% halothane in O₂ and compound or vehicle was administered in a volume of 25 μL solution dropped into each nostril with the dose administered in absolute mg.

Data Analysis: The temperature that produced a half-maximal increase in the tail-withdrawal latency (i.e., T₁₀) was calculated from each temperature-effect curve. The T₁₀ was determined by interpolation from a line drawn between the point above and the point below 10 sec on the temperature-effect curve. For these studies, thermal hypersensitivity was defined as a leftward shift in the temperature-effect curve and a decrease in the T₁₀ value. Reversal of thermal hypersensitivity was defined as a return to baseline of the temperature-effect curve and the T₁₀ value and was calculated according to the following equation:

$$\% MPF = \frac{( {{T}_{10}}^{drug + PGE2} ) - ( {{T}_{10}}^{PGE2} )}{( {{T}_{10}}^{baseline} ) - ( {{T}_{10}}^{PGE2} )} \times 100$$

in which T₁₀^{drug+PGE2} is the T₁₀ after a drug in combination with PGE₂, T₁₀^{PGE2} is the T₁₀ after PGE₂ alone, and T₁₀^{baseline} is the T₁₀ under control conditions. A % MPE value of 100 indicates a complete return to the baseline thermal sensitivity observed without the PGE₂ injection. A value of greater than 100% indicates that the compound tested reduced thermal sensitivity more than the baseline thermal sensitivity without the PGE₂ injection.

Results: Under baseline conditions, maximal tail-withdrawal latencies (i.e., 20 sec) were typically obtained with temperatures of 38, 42, and 46° C. When the water temperature was increased to 50° C., tail-withdrawal latencies for individual rats were typically between 5 and 15 sec. The highest temperature of 54° C. produced tail-withdrawal latencies below 10 sec in all rats. Average baseline T₁₀ values (withdrawal in 10 seconds) were between 49° C. and 51° C.

A dose of 0.1 mg PGE₂ produced a dose- and time-dependent thermal hypersensitivity manifested as a leftward shift in the temperature-effect curve and a decrease in the T₁₀ value. Maximal decreases in tail-withdrawal latencies occurred 15 min after administration, and latencies returned to baseline by 120 min after injection.

Table 3 below shows the effects of PGE₂ in combination with [2-(8,9-Dioxo-2,6-diazabicyclo[5.2.0]non-1 (7)-en-2-yl)ethyl]phosphonic acid (Compound A). Compound A produced a 79% reversal following IP administration (Comparative Example 1) at 10 mg/kg and a 87% reversal following PO administration (Comparative Example 2) at 100 mg/kg. Following IN administration, doses of 0.3 mg, 1 mg and 3 mg produced a 13%, 37% and a 79% reversal, respectively. Based on mg/kg calculations, this represents doses (±SEM) of 0.78 (±0.02), 2.59 (±0.08) and 7.6 (±0.28) mg/kg (Example 8).

TABLE 3
Results of PGE₂-induced thermal hypersensitivity

| | | | % MPE | | | | |
|---|---|---|---|---|---|---|---|
| | Compound | Method | Dose (mg/kg) | | | | |
| Example | tested | of Admin. | 1 | 3 | 10 | 30 | 100 |
| Comparative | A | IP | −7% | 66% | 79% | | |
| Ex. 1 | | | | | | | |
| Comparative | A | PO | | | 5% | 23% | 87% |
| Ex. 2 | | | | | | | |
| Example 8 | A | IN | 13%* | 37%** | 79%*** | | |

*displayed in column for approximation; actual mean dose is 0.8 mg/kg
**displayed in column for approximation; actual mean dose is 2.6 mg/kg
***displayed in column for approximation; actual mean dose is 7.6 mg/kg

In a rat Prostaglandin E₂-induced thermal hypersensitivity model, the in vivo efficacy of intranasal and intraperitoneal administration was found to be similar and ten folds higher than that from the oral administration of Compound A.

1. A pharmaceutical composition for intranasal administration comprising:
a) a therapeutically effective amount of at least one compound of formula (I) or a pharmaceutically acceptable salt thereof: 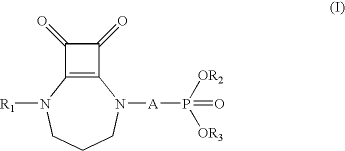
wherein:
R₁ is hydrogen;
A is —(CH₂)ₙ—, where n is 2; and
R₂ and R₃ are hydrogen; and
b) one or more pharmaceutically acceptable additives for forming a composition for intranasal administration.

2. The composition of claim 1 wherein the composition has a pH ranging from about 3 to about 9.

3. The composition of claim 1 wherein the pharmaceutically acceptable additives comprise at least one additive selected from liquid or solid carriers; absorbance enhancers; pH adjusting agents; metal chelating agents; buffers; thickening agents; humectants; or bioadhesives or combinations thereof.

4. The composition of claim 3 wherein the pharmaceutically acceptable additives are present in the composition in an amount of from about 0.25 to about 95 weight percent, based on the total weight of the composition.

5. The composition of claim 1 wherein the composition is in the form of a liquid selected from a solution, gel, suspension, dispersion or emulsion.

6. The composition of claim 5 wherein the compound of formula (I) is present in the composition in an amount of from about 50 mg/ml to about 300 mg/ml.

7. The composition of claim 1 wherein the composition is in the form of a powder.

8. The composition of claim 7 wherein the powder has a particle size of less than about 250 microns.

9. The composition of claim 8 wherein the powder has a particle size of less than about 180 microns.

10. A method for treating at least one condition in a mammal selected from a cerebral vascular disorder selected from cerebral ischemia, cerebral infarction or cerebral vasospasm; cerebral trauma; muscular spasm; a convulsive disorder selected from epilepsy or status epilepticus; hypoglycemia; cardiac arrest; asphyxia anoxia; or spinal chord injury comprising administering intranasally to a mammal in need thereof a therapeutically effective amount of the composition of claim 1.

11. The method of claim 10 wherein the mammal is human.

12. A method for treating at least one condition in a mammal selected from glaucoma or diabetic end organ complications comprising administering intranasally to a mammal in need thereof a therapeutically effective amount of the composition of claim 1.

13. A method for treating at least one condition in a mammal selected from anxiety disorders; mood disorders; schizophrenia; schizophreniform disorder; or schizoaffective disorder comprising intranasally administering to a mammal in need thereof a therapeutically effective amount of the composition of claim 1.

14. The method of claim 13 wherein the anxiety disorder is selected from panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; or the mood disorder is selected from bipolar disorders, depressive disorders selected from major depressive disorder, dysthymic disorder, or substance-induced mood disorder, or mood episodes selected from major depressive episode, manic episode, mixed episode, or hypomanic episode.

15. The method of claim 13 wherein the mammal is human.

16. A method for treating at least one neurodegenerative disorder in a mammal selected from Huntingdon's disease, Alzheimer's disease, amyotrophic lateral sclerosis, chronic dementia, or cognitive impairment comprising intranasally administering to a mammal in need thereof a therapeutically effective amount of the composition of claim 1.

17. The method of claim 16 wherein the mammal is a human.

18. A method for treating Parkinson's disease comprising intranasally administering to a mammal in need thereof a therapeutically effective amount of the composition of claim 1.

19. The method of claim 18 wherein the mammal is a human.

20. A method for treating at least one condition in a mammal selected from inflammatory diseases; fibromyalgia; complications from herpes zoster; prevention of tolerance to opiate analgesia; or withdrawal symptoms from addictive drugs comprising intranasally administering to a mammal in need thereof a therapeutically effective amount of the composition of claim 1.

21. A method for treating pain in a mammal comprising administering intranasally to a mammal in need thereof a therapeutically effective amount of the composition of claim 1.

22. The method of claim 21 wherein the pain is at least one of neuropathic pain; cancer pain; visceral pain associated with pancreatitis or abdominal, pelvic or perineal regions; musculoskeletal pain associated with lower or upper back, spine, fibromylagia, temporomandibular joint, or myofascial pain syndrome; bony pain associated with bone or joint degenerating disorders; headaches; or pain associated with infections, sickle cell anemia, autoimmune disorders, multiple sclerosis, dental procedures, burns or inflammation.

23. The method of claim 21 wherein the mammal is human.

24. The method of claim 21 wherein the pain comprises neuropathic pain and is associated with at least one of diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury selected from phantom limb pain, reflex sympathetic dystrophy or postthoracotomy pain, cancer, chemical injury, toxins, nutritional deficiencies, or viral or bacterial infections.

25. The method of claim 21 further comprising administering a therapeutically effective amount of at least one pain relieving agent.

26. A pharmaceutical composition for intranasal administration, in unit dosage or multiple dose form, comprising:
a) a therapeutically effective unit dosage or multiple dose for intranasal administration of [2-(8,9-dioxo-2,6-diazabicyclo[5.2.0]non-1 (7)-en-2-yl)ethyl] phosphonic acid; and
b) one or more pharmaceutically acceptable additives for forming a composition for intranasal administration.

27. A pharmaceutical composition for intranasal administration comprising:
a) a therapeutically effective amount of at least one compound of formula (I) or a pharmaceutically acceptable salt thereof: 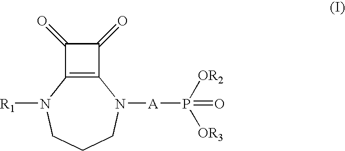
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylene of 1 to 4 carbon atoms or alkenylene of 2 to 4 carbon atoms;
R₂ and R₃ are independently selected from hydrogen, or 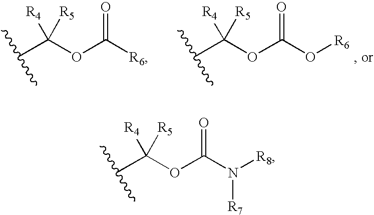
with the proviso that at least one of R₂ and R₃ is not hydrogen;
R₄ and R₅ are independently selected from hydrogen, a C₁ to C₄ alkyl group, a C₅ to C₇ aryl group, a C₆ to C₁₅ alkylaryl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety; a 5 to 13 membered heteroaryl group, a 6 to 21 membered alkylheteroaryl group having 5 to 13 members in the heteroaryl moiety, a C₄ to C₈ cycloalkyl group, a C₅ to C₁₆ alkylcycloalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety, a 5 to 13 membered heteroaryl group, a 6 to 21 membered alkylheteroaryl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halogen atom, a cyano, nitro or hydroxyl group, a C₁-C₆ alkyl group, or a C₁-C₆ alkoxy group; and
b) one or more pharmaceutically acceptable additives for forming a composition for intranasal administration.

28. The composition of claim 27 wherein R₁ is H.

29. The composition of claim 28 wherein A is an alkylene group having the formula —(CH₂)ₙ—, where n is 1 to 3.

30. The composition of claim 29 wherein n is 2.

31. The composition of claim 30 wherein R₄ and R₅ are independently selected from H or a C₁ to C₄ alkyl group, and R₆ is selected from a C₃ to C₁₀ linear or branched alkyl group, a C₅ to C₇ aryl group, a 5- to 7-membered heteroaryl group, or a cycloalkyl group having in the ring 5 to 7 carbon atoms.

32. The composition of claim 31 wherein R₆ is a C₅ to C₇ aryl group.

33. The composition of claim 27 wherein at least one compound of formula (I) is selected from:
a) 3-{2-[8,9-dioxo-2,6-diazabicyclo[5.2.0]non-1 (7)-en-2-yl]ethyl}-3-oxido-7-oxo-7-phenyl-2,4,6-trioxa-3-phosphahept-1-yl benzoate;
b) 3-{2-[8,9-dioxo-2,6-diazabicyclo[5.2.0]non-1 (7)-en-2-yl]ethyl}-3-oxido-7-oxo-8-propyl-2,4,6-trioxa-3-phosphaundec-1-yl 2-propylpentanoate;
c) 2,2-dimethyl-propionic acid (2,2-dimethyl-propionyloxymethoxy)-[2-(8,9-dioxo-2,6-diaza-bicyclo[5.2.0]non-1(7)-en-2-yl)-ethyl]-phosphinoyloxymethyl ester;
d) 7-cyclohexyl-3-{2-[8,9-dioxo-2,6-diazabicyclo[5.2.0]non-1 (7)-en-2-yl]ethyl}-1,5-dimethyl-3-oxido-7-oxo-2,4,6-trioxa-3-phosphahept-1-yl cyclohexanecarboxylate;
e) 7-cyclohexyl-3-{2-[8,9-dioxo-2,6-diazabicyclo[5.2.0]non-1 (7)-en-2-yl]ethyl}-3-oxido-7-oxo-2,4,6-trioxa-3-phosphahept-1-yl cyclohexanecarboxylate;
f) [2-(8,9-Dioxo-2,6-diaza-bicyclo[5.2.0]non-1-(7)-en-2-yl)-ethyl]-phosphonic acid diisopropoxycarbonyl oxymethyl ester;
g) [2-[8,9-dioxo-2,6-diazabicyclo[5.2.0]non-1 (7)-en-2-yl]ethyl]-phosphonic acid bis[1-(benzoyloxy)ethyl] ester;
h) benzoic acid [2-(8,9-dioxo-2,6-diaza-bicyclo[5.2.0]non-1 (7)-en-2-yl)-ethyl]-hydroxy-phosphinoyloxymethyl ester; or
i) [2-(8,9-Dioxo-2,6-diaza-bicyclo[5.2.0]non-1 (7)-en-2-yl)-ethyl]-phosphonic acid di- dimethylcarbamoyloxymethyl ester;
or a pharmaceutically acceptable salt thereof.

34. The composition of claim 27 wherein the composition has a pH ranging from about 3 to about 9.

35. The composition of claim 27 wherein the pharmaceutically acceptable additives comprise at least one additive selected from liquid or solid carriers; absorbance enhancers; pH adjusting agents; metal chelating agents; buffers; thickening agents; humectants; or bioadhesives or combinations thereof.

36. The composition of claim 35 wherein the pharmaceutically acceptable additives are present in the composition in an amount of from about 0.25 to about 95 weight percent, based on the total weight of the composition.

37. The composition of claim 27 wherein the composition is in the form of a liquid selected from a solution, gel, suspension, dispersion or emulsion.

38. The composition of claim 37 wherein the compound of formula (I) is present in the composition in an amount of from about 50 mg/ml to about 300 mg/ml.

39. The composition of claim 27 wherein the composition is in the form of a powder.

40. The composition of claim 39 wherein the powder has a particle size of less than about 250 microns.

41. The composition of claim 40 wherein the powder has a particle size of less than about 180 microns.

42. A method for treating at least one condition in a mammal selected from a cerebral vascular disorder selected from cerebral ischemia, cerebral infarction or cerebral vasospasm; cerebral trauma; muscular spasm; a convulsive disorder selected from epilepsy or status epilepticus; glaucoma; diabetic end organ complications; hypoglycemia; cardiac arrest; asphyxia anoxia; or spinal chord injury comprising administering intranasally to a mammal in need thereof a therapeutically effective amount of the composition of claim 27.

43. The method of claim 42 wherein the mammal is human.

44. A method for treating at least one condition in a mammal selected from anxiety disorders; mood disorders; schizophrenia; schizophreniform disorder; or schizoaffective disorder comprising intranasally administering to a mammal in need thereof a therapeutically effective amount of the composition of claim 27.

45. The method of claim 44 wherein the anxiety disorder is selected from panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder; or the mood disorder is selected from bipolar disorders, depressive disorders selected from major depressive disorder, dysthymic disorder, or substance-induced mood disorder, or mood episodes selected from major depressive episode, manic episode, mixed episode, or hypomanic episode.

46. The method of claim 44 wherein the mammal is human.

47. A method for treating at least one neurodegenerative disorder in a mammal selected from Parkinson's disease, Huntingdon's disease, Alzheimer's disease, amyotrophic lateral sclerosis, chronic dementia, or cognitive impairment comprising intranasally administering to a mammal in need thereof a therapeutically effective amount of the composition of claim 27.

48. The method of claim 47 wherein the mammal is a human.

49. A method for treating at least one condition in a mammal selected from inflammatory diseases; fibromyalgia; complications from herpes zoster; prevention of tolerance to opiate analgesia; or withdrawal symptoms from addictive drugs comprising intranasally administering to a mammal in need thereof a therapeutically effective amount of the composition of claim 27.

50. A method for treating pain in a mammal comprising administering intranasally to a mammal in need thereof a therapeutically effective amount of the composition of claim 27.

51. The method of claim 50 wherein the pain is at least one of neuropathic pain; cancer pain; visceral pain associated with pancreatitis or abdominal, pelvic or perineal regions; musculoskeletal pain associated with lower or upper back, spine, fibromylagia, temporomandibular joint, or myofascial pain syndrome; bony pain associated with bone or joint degenerating disorders; headaches; or pain associated with infections, sickle cell anemia, autoimmune disorders, multiple sclerosis, dental procedures, burns or inflammation.

52. The method of claim 50 wherein the mammal is human.

53. The method of claim 50 wherein the pain comprises neuropathic pain and is associated with at least one of diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury selected from phantom limb pain, reflex sympathetic dystrophy or postthoracotomy pain, cancer, chemical injury, toxins, nutritional deficiencies, or viral or bacterial infections.

54. The method of claim 50 further comprising administering a therapeutically effective amount of at least one pain relieving agent.

55. A pharmaceutical composition for intranasal administration, in unit dosage or multiple dose form, comprising:
a) a therapeutically effective unit dosage or multiple dose for intranasal administration of at least one compound of formula (I) or a pharmaceutically acceptable salt thereof: 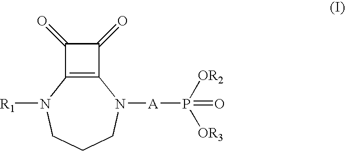
wherein:
R₁ is hydrogen, a C₁ to C₆ alkyl group, a C₂ to C₇ acyl group, a C₁ to C₆ alkanesulfonyl group, or a C₆ to C₁₄ aroyl group;
A is alkylene of 1 to 4 carbon atoms or alkenylene of 2 to 4 carbon atoms;
R₂ and R₃ are independently selected from hydrogen, or 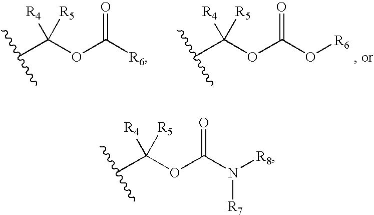
with the proviso that at least one of R₂ and R₃ is not hydrogen;
R₄ and R₅ are independently selected from hydrogen, a C₁ to C₄ alkyl group, a C₅ to C₇ aryl group, a C₆ to C₁₅ alkylaryl group having 5 to 7 carbon atoms in the aryl ring, a C₂ to C₇ alkenyl group, or C₂ to C₇ alkynyl group, or R₄ and R₅ may together form a spiro C₃ to C₈ carbocyclic ring;
R₆ is a C, to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety; a 5 to 13 membered heteroaryl group, a 6 to 21 membered alkylheteroaryl group having 5 to 13 members in the heteroaryl moiety, a C₄ to C₈ cycloalkyl group, a C₅ to C₁₆ alkylcycloalkyl group having 4 to 8 carbon atoms in the cycloalkyl ring;
R₇ and R₈ are independently selected from hydrogen, a C₁ to C₁₂ linear or branched alkyl group, a C₂ to C₇ linear or branched alkenyl or alkynyl group, a C₅ to C₁₃ aryl group, a C₆ to C₂, alkylaryl group having 5 to 13 carbon atoms in the aryl moiety, a 5 to 13 membered heteroaryl group, a 6 to 21 membered alkylheteroaryl group having 5 to 13 members in the heteroaryl moiety, or R₇ and R₈ may together form a cycloalkyl or heterocycloalkyl group having in the ring 4 to 8 carbon atoms and optionally one to two atoms selected from nitrogen, oxygen or sulfur;
wherein any R₁ to R₈ group having an aryl, heteroaryl, cycloalkyl or heterocycloalkyl moiety may optionally be substituted on the aryl, heteroaryl, cycloalkyl or heterocycloalkyl moiety with 1 to about 5 substituents independently selected from a halogen atom, a cyano, nitro or hydroxyl group, a C₁-C₆ alkyl group, or a C₁-C₆ alkoxy group; and
b) one or more pharmaceutically acceptable additives for forming a composition for intranasal administration.

56. The composition of claim 55 wherein at least one of the compound of formula (I) is selected from:
a) 3-{2-[8,9-dioxo-2,6-diazabicyclo[5.2.0]non-1 (7)-en-2-yl]ethyl}-3-oxido-7-oxo-7-phenyl-2,4,6-trioxa-3-phosphahept-1-yl benzoate;
b) 3-{2-[8,9-dioxo-2,6-diazabicyclo[5.2.0]non-1(7)-en-2-yl]ethyl}-3-oxido-7-oxo-8-propyl-2,4,6-trioxa-3-phosphaundec-1-yl 2-propylpentanoate;
c) 2,2-dimethyl-propionic acid (2,2-dimethyl-propionyloxymethoxy)-[2-(8,9-dioxo-2,6-diaza-bicyclo[5.2.0]non-1(7)-en-2-yl)-ethyl]-phosphinoyloxymethyl ester;
d) 7-cyclohexyl-3-{2-[8,9-dioxo-2,6-diazabicyclo[5.2.0]non-1 (7)-en-2-yl]ethyl}-1,5-dimethyl-3-oxido-7-oxo-2,4,6-trioxa-3-phosphahept-1-yl cyclohexanecarboxylate;
e) 7-cyclohexyl-3-{2-[8,9-dioxo-2,6-diazabicyclo[5.2.0]non-1 (7)-en-2-yl]ethyl}-3-oxido-7-oxo-2,4,6-trioxa-3-phosphahept-1-yl cyclohexanecarboxylate;
f) [2-(8,9-Dioxo-2,6-diaza-bicyclo[5.2.0]non-1-(7)-en-2-yl)-ethyl]-phosphonic acid diisopropoxycarbonyl oxymethyl ester;
g) [2-[8,9-dioxo-2,6-diazabicyclo[5.2.0]non-1 (7)-en-2-yl]ethyl]-phosphonic acid bis[1-(benzoyloxy)ethyl] ester;
h) benzoic acid [2-(8,9-dioxo-2,6-diaza-bicyclo[5.2.0]non-1 (7)-en-2-yl)-ethyl]-hydroxy-phosphinoyloxymethyl ester; or
i) [2-(8,9-Dioxo-2,6-diaza-bicyclo[5.2.0]non-1 (7)-en-2-yl)-ethyl]-phosphonic acid di- dimethylcarbamoyloxymethyl ester;
or a pharmaceutically acceptable salt thereof.
